(19) United States
(12) Patent Application Publication
(10) Pub. No.: US 2023/0265416 A1
(43) Pub. Date: Aug. 24, 2023

(54) VIRAL DELIVERY VEHICLE SELECTION

(71) Applicant: Gordian Biotechnology, Inc., San Francisco, CA (US)

(72) Inventors: Martin Borch Jensen, San Francisco, CA (US); Daniel Fuentes, San Francisco, CA (US)

(21) Appl. No.: 17/918,861

(22) PCT Filed: May 10, 2021
(86) PCT No.: PCT/US2021/031592

§ 371 Date: Oct. 13, 2022

Related U.S. Application Data
(60) Provisional application No. 63/023,015, filed on May 11, 2020.
(60) Provisional application No. 63/180,025, filed on Apr. 26, 2021.

(51) Int. Cl. C12N 15/10; C12N 15/86

(52) U.S. Cl. CPC C12N 15/1082; C12N 15/1065; C12N 15/86; C12N 2750/14143

(57) ABSTRACT

Provided herein are libraries of delivery vehicles and methods of uses thereof. The delivery vehicles provided include distinct variants of a virus and nucleic acid sequences encoding a distinct virus-identifying barcode region specific for each virus variants, and a nucleic acid sequence encoding at least one reporter. The methods provided herein include methods of identifying a vehicle effective in targeting a particular cell type.

### CROSS-REFERENCE TO RELATED APPLICATIONS

This application claims the benefit of priority under 35 U.S.C. § 119(e) of U.S. Provisional Application Nos. 63/023,015 filed May 11, 2020 and 63/180,025 filed Apr. 26, 2021. The disclosures of the prior applications are considered part of and are incorporated by reference in the disclosure of this application.

### INCORPORATION OF SEQUENCE LISTING

The material in the accompanying sequence listing is hereby incorporated by reference into this application. The accompanying sequence listing text file, named GORD1130-2WO_SL.txt, was created on May 6, 2021, and is 21 kb. The file can be accessed using Microsoft Word on a computer that uses Windows OS.

### BACKGROUND OF THE INVENTION

### Field of the Invention

The present disclosure relates generally to libraries of delivery vehicles and more specifically to methods of screening of targeted delivery vehicles for identifying delivery vehicles that preferentially target desirable cell types.

PCT/US2019/060144 discloses “compositions and methods of use thereof for screening a plurality of uniquely identifiable therapeutic moiety in vivo by identifying one or more reporters indicative of a cell state.” The '144 PCT contemplates administering a library of a library of expression cassettes to a biological entity (such as an animal or organoid) to identify candidate therapeutic moieties, in some cases using droplet based single cell RNA sequencing.

Davidsson, et al., discloses that “ . . . we have developed a method for capsid engineering named barcoded rational AAV vector evolution (BRAVE), which encompasses all of the benefits of rational design (18, 26, 31-35) while maintaining the broad screening diversity permitted by directed evolution. The key to this method is a viral library production approach where each virus particle displays a protein-derived peptide on the surface, which is linked to a unique barcode in the packaged genome (36). Through hidden Markov model-based clustering (37), we were able to identify consensus motifs for neuronal cell type-specific retrograde transport and expression in the brain. The BRAVE approach enables the selection of functional capsid structures using only a single-generation screening.”

### SUMMARY OF THE DISCLOSURE

The instant disclosure is based at least in part on the discovery that libraries of delivery vectors can be used to identify delivery vehicles that are effective in targeting particular cell types.

In one embodiment, the disclosure provides a library including two or more distinct delivery vehicles, each delivery vehicle including a) a distinct variant of a virus; b) a nucleic acid sequence encoding a distinct virus-identifying barcode region specific for each of the virus variants, wherein the barcode sequence is different than a nucleic acid sequence encoding a protein of the variant of the virus; and c) a nucleic acid sequence encoding at least one reporter, which when expressed in a cell, is indicative of a cell state or a likelihood of a cell state of a cell.

In one aspect, each of the vectors are selected from the group consisting of adeno-associated viruses and lentivirus. In some aspects, the distinct variants of a virus are substituted for distinct variants of lipid nanoparticles. In other aspects, each of the vectors are variants of adeno-associated viruses. In some aspects, the distinct variant of a virus contains a uniquely modified cap gene region linked to the distinct virus-identifying barcode region. In various aspects, the cap gene and distinct virus-identifying barcode regions are isolated using beads affixed with complementary DNA to the distinct virus-identifying barcode region. In one aspect, the regions that were isolated are identified by insertion of the region into a new plasmid; amplification of the new plasmid; and Sanger sequencing of the plasmid. In some aspects, a Polymerase III promotor region is operably linked to the distinct virus-identifying barcode region. In one aspect, a capture sequence is operably linked to the distinct virus-identifying barcode region under the control of the Polymerase III promoter. In some aspects, the capture sequence has a sequence including any one of SEQ ID NOs:1-4. In another aspect, one or more molecular enrichment sequences are operably linked to the distinct virus-identifying barcode region under the control of the Polymerase III promoter. In some aspects, the one or more molecular enrichment sequences have a sequence including any one of SEQ ID NOs:5-84. In one aspect, a unique genome identification (UGI) sequence is operably linked to the distinct virus-identifying barcode region under the control of the Polymerase III promoter. In some aspects, the UGI has a sequence including SEQ ID NO:85. In other aspects, the library includes more than one, about 5 or more; about 50 or more; about 100 or more; about 10,000 or more; about 100,000 or more; about 1,000,000 or more; or about 10,000,000 or more distinct delivery vehicles.

In another embodiment, the disclosure provides a method for identifying a vehicle effective in targeting a particular cell type including administering to an animal or an organoid a library including two or more distinct delivery vehicles, each delivery vehicle including a) a distinct variant of a virus; b) a nucleic acid sequence encoding a distinct virus-identifying barcode region specific for each of the virus variants, wherein the barcode sequence is different than a nucleic acid sequence encoding a protein of the variant of the virus; and c) a nucleic acid sequence encoding at least one reporter, which when expressed in a cell, is indicative of a cell state or a likelihood of a cell state of a cell; obtaining a sample from the animal or organoid to generate a cell population; enriching the cell population for those cells containing a reporter; using single cell sequencing to identify a delivery vehicle that results in a change in a cell state or a likelihood of a cell state of a cell of the animal or the organoid and thereby the vector; and using single cell sequencing to identify the type of cells having the change in cell state and to determine the relative rate of transduction for one of the distinct vectors in the different cell types, thereby identifying the vehicle.

In one aspect, the change in cell state or likelihood of a change in cell state indicates the successful delivery and expression of the nucleic acid sequences to a cell of the cell population after enriching. In another aspect, the cell state or likelihood of cell state is determined by the presence of increased or decreased levels of proteins or nucleic acid sequences. In some aspects, identifying includes using a technique selected from the group consisting of single cell analysis, RNA sequencing, single cell RNA sequencing, droplet-based single cell RNA sequencing, and bulk analysis. In other aspects, identifying includes identifying the delivery vehicle based on the presence of a reporter and vector-identifying barcode within a cell of the cell population after enriching. In some aspects, the identification step further includes identifying the cell type of a cell determined to have been effected by the delivery vehicle. In other aspects, enriching includes using a technique selected from the group consisting of fluorescence automated cell sorting, immunoprecipitation, magnetic immunoprecipitation, flow cytometry, and microfluidic sorting. In one aspect, the fluorescent marker is GFP.

In an additional embodiment, the disclosure provides a method for identifying a vehicle effective in targeting a particular cell type including administering to an animal or an organoid a library including two or more distinct delivery vehicles, each delivery vehicle including a) a distinct variant of an adeno-associated virus; b) a nucleic acid sequence encoding a distinct virus-identifying barcode region specific for each of the virus variants, wherein the barcode sequence is different than a nucleic acid sequence encoding a protein of the variant of the virus; and c) a nucleic acid sequence encoding GFP, which when expressed in a cell, is indicative of successful delivery of the nucleic acid sequences to a cell; obtaining a sample from the animal or organoid to generate a cell population; enriching the cell population for those cells containing GFP; using single cell sequencing to identify a delivery vehicle that results in expression of the nucleic acid sequences with a cell; and using single cell sequencing to identify the type of cells having the change in cell state and to determine the relative rate of transduction for one of the distinct vectors in the different cell types, thereby identifying the vehicle.

In various aspects, the methods described herein further include identifying a type transduced cell, and/or a localization of a transduced cell in a tissue. In some aspects, identifying includes using spatial transcriptomics.

### DETAILED DESCRIPTION OF THE DISCLOSURE

The instant disclosure is based at least in part on the discovery that libraries of delivery vectors can be used to identify delivery vehicles that are effective in targeting particular cell types.

Before the present compositions and methods are described, it is to be understood that this invention is not limited to particular compositions, methods, and experimental conditions described, as such compositions, methods, and conditions may vary. It is also to be understood that the terminology used herein is for purposes of describing particular embodiments only, and is not intended to be limiting, since the scope of the present invention will be limited only in the appended claims.

As used in this specification and the appended claims, the singular forms “a”, “an”, and “the” include plural references unless the context clearly dictates otherwise. Thus, for example, references to “the method” includes one or more methods, and/or steps of the type described herein which will become apparent to those persons skilled in the art upon reading this disclosure and so forth.

All publications, patents, and patent applications mentioned in this specification are herein incorporated by reference to the same extent as if each individual publication, patent, or patent application was specifically and individually indicated to be incorporated by reference.

Unless defined otherwise, all technical and scientific terms used herein have the same meaning as commonly understood by one of ordinary skill in the art to which this invention belongs. Although any methods and materials similar or equivalent to those described herein can be used in the practice or testing of the invention, it will be understood that modifications and variations are encompassed within the spirit and scope of the instant disclosure. The preferred methods and materials are now described.

Despite recent advancements, there remain a number of challenges in gene therapy and other types of clinical interventions, including translation of in vitro research into in vivo therapies, designing therapies when the disease etiology is unknown or not well understood, screening large numbers of viral and non-viral delivery vehicles, and screening vehicles in vivo to account for intracellular and extracellular factors that impact vehicle design, safety, and/or efficacy. Therapies for aging related diseases or conditions can be complex due to multiple pathways and factors, including cellular and environmental factors, that contribute to the disease or condition, and/or involve poorly understood mechanisms.

A major challenge in gene therapy is delivery of therapeutics to diseased tissues and/or cell types, with both high specificity and efficient delivery. One of the most commonly used delivery vectors is adeno-associated viruses. A number of serotypes of this virus have been discovered, with different specificity and efficiency for different tissues and cell types, and new serotypes can be designed in a variety of ways. This includes creation of many (upward of thousands) variant serotypes simultaneously, which creates a need to efficiently characterize the performance of variants.

In the past, and still, this was typically done by injecting one variant of virus, containing a fluorescent marker, into the tissue of interest, and using microscopy and cell-type specific markers to determine which and how many cells the virus transduced. This method is very laborious and limited in throughput. A more efficient version is to use fluorescence activated cell sorting (FACS) to rapidly test for transduction in many cells stained with antibodies identifying cell type. This approach requires cell-type specific surface markers, and also will only work for cell types that survive the sorting process (many neurons, for example, do not).

Another approach to increase efficiency is to include DNA barcodes in the cargo of each virus variant, which allows next-generation sequencing to quantify the amount of each virus type that has entered a tissue or pool of cells. When done on isolated cells, the same restrictions when sorting described above apply. When done using a whole tissue as starting material, little to no knowledge is gained about which type of cell the virus transduced. And in both cases, one learns only the average number of viruses transducing each cell, not the distribution (e.g. a small subset of cells could get transduced heavily).

Certain aspects and embodiments of the present disclosure propose to achieve biological resolution and high-throughput by combining barcodes that are expressed as RNA with single-cell sequencing methods capturing and labeling both cellular and barcode RNA from individual cells. Thus, individual variants of adeno-associated virus may be produced with unique barcodes under strong, universal promoters. Expression of multiple copies of the barcode per viral genome can improve detection rates during single-cell sequencing. Simultaneously, cell type identity of every transduced cell may in some embodiments be determined by low-depth RNA sequencing. This allows testing transduction in all cell types of a given tissue simultaneously, which is valuable for determining specificity. Moreover, single-cell sequencing has proven in many examples more powerful for identifying new cell types/sub-types than using specific markers. This detection power can include specific states of a given cell type relevant for the investigation in question, e.g. cells in a particular diseased state (inflamed, fibrotic, degenerating, etc.), tumor versus non-tumor cells, dividing versus non-dividing cells, activated cells (e.g. neurons), and so on. In many cases, no specific markers can be applied in high throughput to such states.

Accordingly in a first aspect, a method is provided for identifying a vehicle effective in targeting a particular cell type including: administering to an animal or an organoid a library including two or more distinct delivery vehicles, each delivery vehicle including: (a) a distinct variant of a virus; (b) a nucleic acid sequence encoding a distinct virus-identifying barcode region specific for each of the virus variants, wherein the barcode sequence is different than a nucleic acid sequence encoding a protein of the variant of the virus; and (c) a nucleic acid sequence encoding at least one reporter, which when expressed in a cell, is indicative of a cell state or a likelihood of a cell state of a cell; obtaining a sample from the animal or organoid to generate a cell population; enriching the cell population for those cells containing a reporter; using single cell sequencing to identify a delivery vehicle that results in a change in a cell state or a likelihood of a cell state of a cell of the animal or the organoid and thereby the vector; and using single cell sequencing to identify the type of cells having the change in cell state and to determine the relative rate of transduction for one of the distinct vectors in the different cell types, thereby identifying the vehicle.

In a second aspect, provided is a library including two or more distinct delivery vehicles, each delivery vehicle including: (a) a distinct variant of a virus; (b) a nucleic acid sequence encoding a distinct virus-identifying barcode region specific for each of the virus variants, wherein the barcode sequence is different than a nucleic acid sequence encoding a protein of the variant of the virus; and (c) a nucleic acid sequence encoding at least one reporter, which when expressed in a cell, is indicative of a cell state or a likelihood of a cell state of a cell.

Provided herein are methods and compositions to improve code-correction. As such, some aspects and embodiments presented include multiple (e.g., three, four, five, or more than five) virus-identifying barcodes, each identifying a same virus variant. During droplet-based single cell sequencing, it is possible for oligonucleotides from one cell to be mislabeled as another cell, or for fragments of one cell to attach to and contaminate another cell. Use of a single barcode per virus variant may make it difficult or impossible to distinguish between: (1) contaminating barcodes, and (2) a cell receiving multiple variants of a virus and expressing each of the pertinent barcodes. Conversely, if a triplet of barcodes describes a single virus variant, detection of individual components of the triplet can be identified as likely contamination, whereas detection of the entire triplet occurring alongside a separate unique triplet allows identification of cells having received multiple unique virus variants. Inclusion of multiple barcodes to identify a single virus variant reduces the risk of template switching significantly, which reduces the likelihood of misidentification of the virus variant received by a cell.

The term “barcode,” as used herein, generally refers to a label, or identifier, that conveys or is capable of conveying information about the analyte. A barcode can be part of an analyte. A barcode can be a tag attached to an analyte (e.g., nucleic acid molecule) or a combination of the tag in addition to an endogenous characteristic of the analyte (e.g., size of the analyte or end sequence(s)). A barcode may be unique. Barcodes can have a variety of different formats, for example, barcodes can include polynucleotide barcodes; random nucleic acid and/or amino acid sequences; and synthetic nucleic acid and/or amino acid sequences. A barcode can be attached to an analyte in a reversible or irreversible manner. A barcode can be added to, for example, a fragment of a deoxyribonucleic acid (DNA) or ribonucleic acid (RNA) sample before, during, and/or after sequencing of the sample. Barcodes can allow for identification and/or quantification of individual sequencing-reads in real time. In some cases, the barcode may be a virus variant specific barcode. In some aspects, the first two nucleotides of a barcode are a ‘GG’.

A “reporter gene” as used herein refers to any sequence that produces a protein product that can be measured, preferably, although not necessarily in a routine assay (i.e., a reporter). Suitable reporter genes include, but are not limited to, sequences encoding proteins that mediate antibiotic resistance (e.g., ampicillin resistance, neomycin resistance, G418 resistance, puromycin resistance), sequences encoding colored or fluorescent or luminescent proteins (e.g., green fluorescent protein (GFP), enhanced green fluorescent protein (eGFP), red fluorescent protein, luciferase), and proteins which mediate enhanced cell growth and/or gene amplification (e.g., dihydrofolate reductase). Epitope tags include, for example, one or more copies of FLAG, His, myc, Tap, HA or any detectable amino acid sequence. “Expression tags” include sequences that encode reporters that may be operably linked to a desired gene sequence in order to monitor expression of the gene of interest. In some cases, a reporter may be the protein product of a reporter gene.

In various embodiments described herein, the reporter used in GFP. The term GFP as used herein is meant to generally refer to both the wild type GFP, as purified from the jellyfish Aequorea Victoria, and any of the GFP derivatives that have been discovered and/or engineered since to display improved spectral characteristics of GFP, resulting in increased fluorescence, photostability, and a shift of the major excitation peak to 488 nm, with the peak emission kept at 509 nm, for example. GFP can refer to a 37° C. folding efficiency (F64L) point mutant, yielding enhanced GFP (EGFP), and which has an extinction coefficient (denoted ε) of 55,000 M−1 cm−1. [20] The fluorescence quantum yield (QY) of EGFP is 0.60. The relative brightness, expressed as ε·QY, is 33,000 M−1 cm−1. In various embodiments described herein, the reporter is GFP, such as eGFP.

In some aspects, the distinct virus-identifying barcode is operably linked to a promotor region, and to one or more additional sequences.

Most droplet-based single-cell sequencing systems capture polyadenylated transcripts exclusively, and therefore previous pooled screens express barcodes from polymerase II promoters (Pol II). Polymerase III promoters (Pol III) have much stronger expression (˜10×) than Pol II, but the resulting transcripts are not polyadenylated. Recent work has led to the inclusion of specific features (capture sequences) in single-cell sequencing systems to preferentially capture barcode RNA (Replogle 2018). Thus, in some embodiments, the compositions and methods provided herein combine Pol III driven therapeutic moiety barcodes with capture sequence systems, circumventing the need to capture polyadenylated sequences and increasing the amounts of capture sequences and virus-identifying barcodes. As used herein, “operably linked”, “operable linkage”, “operatively linked”, or grammatical equivalents thereof refer to juxtaposition of genetic elements, e.g., a promoter, an enhancer, a polyadenylation sequence, etc., wherein the elements are in a relationship permitting them to operate in the expected manner. For instance, a promoter is operatively linked to a coding region if the promoter helps initiate transcription of the coding sequence. There may be intervening residues or elements between the promoter and coding region, such as an enhancer, so long as this functional relationship is maintained.

In certain embodiments, the system includes multiple copies of the Pol III driven barcodes with the capture sequence systems, thereby further increasing the number of transcripts. The term “PolIII/therapeutic moiety barcode/capture element” or “P3TM element”, as used herein, refers to a nucleic acid sequence of an expression cassette including a PolIII promoter operably linked to at least one virus variant barcode and one or more additional sequences that may optionally include a capture sequence. In various embodiments, the increase in number of barcode and capture sequence transcripts may improve the barcode capture efficiency and offer the ability to detect sequencing errors through code-correction, as they will be identifiable as having come from the same cell.

In some aspects and embodiments, a nucleic acid sequence encoding a virus-identifying barcode region that is operably linked to a PolIII promoter, included for example in a P3TM element, includes a virus-identifying barcode and optionally additional sequences controlled by the PolIII promoter.

In some aspects and embodiments, a sequence of an expression cassette that is operably linked to a PolIII promoter as provided herein (e.g., a P3TM element) includes a virus-identifying barcode and optionally additional sequences controlled by the PolIII promoter; wherein said optional additional sequences controlled by the PolIII promoter include one or more sequences selected from the group consisting of a capture sequence; a molecular enrichment sequences; and a unique genome identification (UGI) sequence. In some aspects and embodiments, a sequence of an expression cassette that is operably linked to a PolIII promoter as provided herein (e.g., a P3TM element) includes a virus-identifying barcode and optionally additional sequences controlled by the PolIII promoter; wherein said optional additional sequences controlled by the PolIII promoter include one or more capture sequences such as provided herein. In certain embodiments, a capture sequence as provided herein is at or near the 3′ end of the P3TM element. In some aspects and embodiments, a sequence of an expression cassette that is operably linked to a PolIII promoter as provided herein (e.g., a P3TM element) includes a virus-identifying barcode and optionally additional sequences controlled by the PolIII promoter; wherein said optional additional sequences controlled by the PolIII promoter include one or more molecular enrichment sequences such as provided herein. In some aspects and embodiments, a sequence of an expression cassette that is operably linked to a PolIII promoter as provided herein (e.g., a P3TM element) as provided herein includes a virus-identifying barcode and optionally additional sequences controlled by the PolIII promoter; wherein said optional additional sequences controlled by the PolIII promoter include one or more unique genome identification (UGI) sequences such as provided herein. In some embodiments, a P3TM of the disclosure (including a virus-identifying barcode and optionally one or more of a capture sequence; a molecular enrichment sequence; and a unique genome identification (UGI) sequence) is 50-500 bases; or 50-250 bases; or 75-200 bases; or 75-100 bases; or 100-150 bases; or 120-130 bases; or about 100 bases; or about 110 bases; or about 120 bases; or about 125 bases; or about 130 bases; or about 140 bases; or about 150 bases in length. In some embodiments, a therapeutic moiety barcode operably linked to a PolIII promoter (e.g., within the P3TM element) is 5-50 bases; or 10-30 bases; or 12-28 bases; or 14-26 bases; or 15-25 bases; or 16-24 bases; or 17-23 bases; or 18-22 bases; or 19-21 bases; or about 15 bases; or about 16 bases; or about 17 bases; or about 18 bases; or about 19 bases; or about 20 bases; or about 21 bases; or about 22 bases; or about 23 bases; or about 24 bases; or about 25 bases in length.

As used herein, the term “polymerase III promoter” or “Pol III promoter” refers to a DNA sequence that recruits and enables initiation of transcription by RNA polymerase III (e.g., U6 promoter). These promoters allow the transcription of the downstream sequences relative to the promotor region.

The term “capture sequence” as used herein refers to a nucleic acid sequence appended to an expressed oligonucleotide, which nucleic acid sequence is reverse complementary to an oligonucleotide sequence present on the surface of beads used in droplet based single-cell sequencing. This capture sequence allows the expressed oligonucleotides to be captured onto the beads and enter the single cell sequencing workflow, in the absence of polyadenylation of the expressed oligonucleotide. In some aspects or embodiments, a capture sequence includes a sequence selected from the group consisting of 5′-GCTTTAAGGCCGGTCCTAGCAA-3′ (SEQ ID NO: 1) and 5′-GCTCACCTATTAGCGGCTAAGG-3′ (SEQ ID NO: 2). In some embodiments, the methods involve capture using an oligonucleotide ‘spike’ that is complementary to 10× reagents and any target sequence within the P3TM element, for example as described in Replogle et al., Nature Biotechnology (doi.org/10.1038/s41587-020-0470-y). In such embodiments SEQ ID NO: 1 or 2 may not be necessary as capture sequence. Exemplary spike oligonucleotides include SEQ ID NOs:3 and 4. In some aspects, a capture sequence can be replaced by a spike oligonucleotide for the capture of the target sequences. In other aspects, a capture sequence and a spike oligonucleotide can be used for the capture of the target sequences.

The term “molecular enrichment sequence” as used herein refers to a sequence, often operably linked to a PolIII promoter (for example a sequence within a P3TM element), that may in certain embodiments act to increase the amount of virus-identifying barcode that is captured, identified and/or measured in methods provided herein by increasing expression, stability, and/or capture of the virus-identifying barcode molecules.

In some embodiments, a molecular enrichment sequence is, or includes, the sequence: CTTGGATCGTACCGTACGAA (SEQ ID NO: 5). In some embodiments a molecular enrichment sequence is, or includes, the sequence: SEQ ID NO:5; wherein the sequence starts within 10 bases; or 8 bases; or 5 bases; or 4 bases; or 3 bases; or two bases; or one base of the transcription starting site. In other embodiments, a molecular enrichment sequence as provided herein includes the sequence CCCCNN (SEQ ID NO:6) or NNCCCC (SEQ ID NO:7). In some embodiments, a molecular enrichment sequence as provided herein includes SEQ ID NO:6 or 7, located in a region having a low probability of forming a secondary structure. In some embodiments, the molecular enrichment sequence includes repeats, such as 1 repeat; or 2 repeats; or 3 repeats; or 4 repeats; or 5 repeats; or more repeats of SEQ ID NO:6 or 7. In some embodiments, the molecular enrichment sequence includes repeats, such as 1 repeat; or 2 repeats; or 3 repeats; or 4 repeats; or 5 repeats; or more repeats of SEQ ID NO:6; and wherein the repeats are located in a region having a low probability of forming a secondary structure.

In some embodiments, the molecular enrichment sequence includes one or more sequences selected from SEQ ID NOs:8-54. In some embodiments, a molecular enrichment sequence (which may be included in a P3TM element) is, or includes, any one of SEQ ID NOs:8-54, wherein the sequence starts within 10 bases; or 8 bases; or 5 bases; or 4 bases; or 3 bases; or two bases; or one base of the transcription starting site.

In other embodiments, a molecular enrichment sequence is, or includes, a sequence reading as follows: (1-3 Gs)(optional A)(1-2 Cs)(A/T)(A/T). In some embodiments, the first nucleotide of a transcription starting site of a sequence driven by a PolIII promotor (such as a P3TM element) is a ‘G’. In some embodiments, the first two nucleotides of a transcription starting site of a sequence driven by a PolIII promotor (such as a P3TM element) is a ‘GG’. In some embodiments, a molecular enrichment sequence (for example in a P3TM element) is, or includes, a sequence reading as follows: (1-3 Gs)(optional A)(1-2 Cs)(A/T)(A/T); wherein the sequence starts within 10 bases; or 8 bases; or 5 bases; or 4 bases; or 3 bases; or two bases; or one base of the transcription starting site. In some embodiments, the molecular enrichment sequence includes one or more sequences selected from SEQ ID NOs:55-84. In some embodiments, a molecular enrichment sequence (for example included in a P3TM element) is, or includes, any one of SEQ ID NOs:55-84, wherein the sequence starts within 10 bases; or 8 bases; or 5 bases; or 4 bases; or 3 bases; or two bases; or one base of the transcription starting site. In some embodiments, a molecular enrichment sequence (for example included in a P3TM element) is, or includes, any one of SEQ ID NOs:5-84, wherein the sequence starts within 10 bases; or 8 bases; or 5 bases; or 4 bases; or 3 bases; or two bases; or one base of the transcription starting site.

The term “unique genome identification (UGI) sequence” refers to a sequence that is introduced into an expression cassette (e.g., into a P3TM element) and is unique to a particular plasmid or virus clone in a library. In various embodiments of the methods provided herein, the UGI sequence can be used to quantify the amount of a particular plasmid or virus clone that delivers a particular therapeutic intervention into a cell. In various embodiments, the nucleotide sequence of UGIs as provided herein may be randomly generated. In some embodiments a UGI sequence is 5-25 bases or 5-20 bases; or 5-15 bases; or 5-12 bases; or 5-10 bases; or 6-10 bases; or about 5 bases; or about 6 bases; or about 7 bases; or about 8 bases; or about 9 bases; or about 10 bases; or about 11 bases; or about 12 bases; or about 13 bases; or about 14 bases; or about 15 bases in length.

In a third aspect, a method is provided for identifying a vehicle effective in targeting a particular cell type including: administering to an animal or an organoid a library including two or more distinct delivery vehicles, each delivery vehicle including: (a) a distinct variant of an adeno-associated virus; (b) a nucleic acid sequence encoding a distinct virus-identifying barcode region specific for each of the virus variants, wherein the barcode sequence is different than a nucleic acid sequence encoding a protein of the variant of the virus; and (c) a nucleic acid sequence encoding GFP, which when expressed in a cell, is indicative of successful delivery of the nucleic acid sequences to a cell; obtaining a sample from the animal or organoid to generate a cell population; enriching the cell population for those cells containing GFP; using single cell sequencing to identify a delivery vehicle that results in expression of the nucleic acid sequences with a cell; and using single cell sequencing to identify the type of cells having the change in cell state and to determine the relative rate of transduction for one of the distinct vectors in the different cell types.

The methods provided herein can further include the identification of the type of cells that are transduced, and/or the localization of the transduced cells within a tissue. Identifying the cell type transduced by a certain virus variant, and the anatomical location of said transduced cells can be used to reveal information about the virus' ability to transduce cells near certain anatomical features. For example, identifying transduced cells type and/or localization can indicate a virus ability to transduce blood vessel cells, tumor cells, cells in fibrotic regions, etc., and/or cells around such cell types. Identifying transduced cells type and/or localization can be accomplished, for example, by using spatial transcriptomics as the single cell sequencing modalities in screens described above. As used herein, the term “spatial transcriptomics” refers to the molecular assay that is performed to identify a type and/localization of a transduced cells. In some aspects, spatial transcriptomics may involve placing two-dimensional tissue section on a coated surface (such as a glass slide) covered with ‘surface probes’ and subsequently initiating a reverse transcription reaction to label mRNA molecules in the tissue section with two barcodes. In various aspects, the barcode includes nucleotides. A first barcode can be used to identify the individual mRNA molecules, and a second barcode can contain two-dimensional coordinates. This allows for subsequent reverse transcription, amplification, and next-generation sequencing of the tissue-derived cDNA, while preserving information about the original mRNA molecule and its location in the tissue. Barcode molecules identifying specific virus variants can be sequenced any single cell sequencing methods known in the art. In some embodiments, an imaging step is performed before the reverse transcription step that can be used to correlate the spatial coordinates identified by the spatial barcode surface probes. In some embodiments, the tissue is stained using chemical, antibodies or other indicators of specific cellular states, for example the presence or concentration of specific proteins in a cell.

In certain aspects and embodiments, each of the distinct variants of a virus are selected from the group consisting of adeno-associated viruses and lentivirus. In certain aspects and embodiments, each of the distinct variants of a virus are a variant of an adeno-associated virus. In certain aspects and embodiments, the variants of a virus are substituted for variants of lipid nanoparticles. In certain aspects and embodiments, the change in cell state or likelihood of a change in cell state indicates the successful delivery and expression of the nucleic acid sequences to a cell of the cell population after the enriching. In certain aspects and embodiments, the cell state or likelihood of cell state is determined by the presence of increased or decreased levels of proteins or nucleic acid sequences.

In certain aspects and embodiments, the identifying includes a technique selected from the group consisting of single cell analysis, RNA sequencing, single cell RNA sequencing, droplet-based single cell RNA sequencing, spatial transcriptomics, bulk analysis. In certain aspects and embodiments, the identifying step includes identifying the delivery vehicle based on the presence of a reporter and vector-identifying barcode within a cell of the cell population after the enriching. In certain aspects and embodiments, the identification step further includes identifying the cell type of a cell determined to have been effected by the delivery vehicle.

In certain aspects and embodiments, the enriching includes a technique selected from the group consisting of fluorescence automated cell sorting, immunoprecipitation, magnetic immunoprecipitation, flow cytometry, and microfluidic sorting. In certain aspects and embodiments, the reporter is a fluorescent marker. In certain aspects and embodiments, the fluorescent marker is GFP.

In certain aspects and embodiments, the distinct variant of a virus contains a uniquely modified cap gene region linked to the distinct virus-identifying barcode region. In certain aspects and embodiments, the cap gene and distinct virus-identifying barcode regions are isolated using bead affinity assays. In certain aspects and embodiments, the isolated regions are identified by insertion into a new plasmid; and amplification of the newly generated plasmid; and Sanger sequencing of the plasmid. In certain aspects and embodiments, a Polymerase III promoter region is operably linked to the distinct virus-identifying barcode region.

In certain aspects and embodiments, the library includes more than one, about 5 or more distinct delivery vehicles. In certain aspects and embodiments, the library includes 50 or more distinct delivery vehicles. In certain aspects and embodiments, the library includes 100 or more distinct delivery vehicles. In certain aspects and embodiments, the library includes 10,000 or more distinct delivery vehicles. In certain aspects and embodiments, the library includes 100,000 or more distinct delivery vehicles. In certain aspects and embodiments, the library includes 1,000,000 or more distinct delivery vehicles. In certain aspects, the library includes 10,000,000 or more distinct delivery vehicles.

A variant of this approach that may be used in some embodiments is to express both a constant fluorescent protein and a unique expressed barcode in the virus. This allows sorting of only transduced cells, reducing overall sequencing burden/cost. Unlike the FACS analysis described above, this approach does not require prior knowledge or preservation of cell type markers and can sort and sequence nuclei instead of whole cells for cell types not amenable to FACS. A further variant contemplated herein involves using custom protein tags targetable by magnetic bead-conjugated antibodies can be expressed in the nuclear or cellular membrane, to allow magnetic separation instead of sorting by fluorescence. Both of these methods may require removing the viral genes used to generate variants from the inter-ITR region of DNA that is packaged into viral capsids. Doing so requires, in some embodiments, the expressed barcodes to be identifiably linked to capsid variants. In order to profile many variants, next-generation sequencing is typically used. This technology often has a limit in the length of sequences it can read, and that length may be shorter than the length of the cap gene varied to create variant viruses. As a result, most approaches to creating barcoded variants vary only within a small region near either end of the cap region, close enough to the barcode to be measured by NGS. Bulk sequencing has been the primary means within many of these library-style screening paradigms thus far. However, many current sequencing methods encounter read-length and reliability limitations, which restrict the viable regions for modification of the capsids to a small portion near either end of the gene, preventing discovery of new variants with desirable properties containing modifications outside of these end regions. Various aspects and embodiments of methods provided herein further contemplate a method whereby a large pooled library of capsid variants is created by methods that do not preserve complete information of the variants contained, such as DNA shuffling, and coupling each variant to a random barcode contained on the same plasmid, which gets packaged into the viral genome and can be expressed by a promoter for single-cell sequencing readouts described above. Once the barcode of variants with desired tropism or other features is identified, restriction sites and/or recombination may be used on the DNA library (or amplified product thereof) used to produce virus to extract smaller DNA fragments containing barcodes and complete cap genes. These fragments are captured using oligonucleotides complementary to the barcode (which was identified in sequencing), attached to a surface or bead. Thereby, the fragments matching the barcode identified from single-cell sequencing can be extracted from the mixed pool and cloned into a new plasmid that can be Sanger sequenced to identify the corresponding cap variant. This method is particularly suited to testing variants of AAV but may be contemplated for other viruses.

The disclosure can in certain embodiments be equally applied to other types of gene delivery systems that accept custom nucleotide cargo, including but not limited to lentivirus, adenovirus, exosomes/extracellular vesicles, and lipid nanoparticles.

The methods herein can in some embodiments be applied by linking barcodes to promotor variants within the plasmid cargo of vehicles, rather than cap region variants of vehicles. In addition to delivery vehicles, the methods can also be applied to promoters intended to express gene therapies or other nucleotide cargo in target cells. In this version, one or more delivery vehicles known or suspected to target cell types of interest contain cargoes with variations of one or more promoters, each expressing a unique barcode as above. The level of barcode expression in different cell types thus describes both strength and specificity of the promoter variant. Here, the coupling of expression readouts with single-cell sequencing of cell states offers the further advantage of identifying not only average expression of the promoter, but variation of expression in specific cell states (such as those described above, e.g. promoters changing in disease or during specific cellular programs). Examples of promoter variants include, but is not limited to: different endogenous promoters or enhancers, synthetic promoters designed rationally (e.g. from known binding motifs) or through directed evolution, smaller fragments of endogenous or synthetic promoters, combinations of promoters and/or enhancers (e.g. strong universal promoters and cell type specific enhancers), synthetic promoters containing fragments of multiple endogenous or synthetic promoters. The system could also measure additivity/synergy of including multiple promoters in the same gene therapy delivery vehicle.

Accordingly in an additional aspect, a method is provided for identifying a promotor region effective in a particular cell type including: administering to an animal or an organoid a library including two or more distinct delivery vehicles, each delivery vehicle including: (a) a viral vector; (b) a nucleic acid sequence encoding a unique promotor region operably linked to a distinct promotor-identifying barcode region specific for the unique promotor region, wherein the barcode sequence is different than a nucleic acid sequence encoding a protein of the variant of the virus; and (c) a nucleic acid sequence encoding at least one reporter, which when expressed in a cell, is indicative of successful delivery and expression of the nucleic acid sequences of a delivery vehicle; obtaining a sample from the animal or organoid to generate a cell population; enriching the cell population for those cells containing a reporter; using single cell sequencing to identify a delivery vehicle that results in a change in a cell state or a likelihood of a cell state of a cell of the animal or the organoid and thereby expression of the delivery vehicle; and using single cell sequencing to identify the type of cells having the change in cell state and to determine the relative rate of transduction for one of the distinct promotor regions in the different cell types.

In any of the aspects or embodiments, the methods of enriching the cell populations using sequencings can be performed, for example, from the methodology as described in PCT/US2019/060144, hereby incorporated by reference in its entirety.

Presented below are examples discussing delivery vehicle libraries contemplated for the discussed applications. The following examples are provided to further illustrate the embodiments of the present invention but are not intended to limit the scope of the invention. While they are typical of those that might be used, other procedures, methodologies, or techniques known to those skilled in the art may alternatively be used.

### EXAMPLES

### Example 1

### Constructing a Delivery Vehicle Library

A delivery vehicle library is constructed using fragmentation-and-recombination-based DNA shuffling, along similar lines to Grimm D, Lee J S, Wang L, et al. In vitro and in vivo gene therapy vector evolution via multispecies interbreeding and retargeting of adeno-associated viruses. J Virol. 2008; 82(12):5887-5911. doi:10.1128/JVI.00254-08 (www.ncbi.nlm.nih.gov/pmc/articles/PMC2395137/) and Herrmann et al. A Robust and All-Inclusive Pipeline for Shuffling of Adeno-associated Viruses. ACS Synth. Biol. 2019, 8, 1, 194-206 Dec. 4, 2018 (pubs.acs.org/doi/10.1021/acssynbio.8b00373). The following distinctions to the protocols are included: A plasmid library containing the input viral genes is DNA shuffled as described. The resulting shuffled library of capsids is inserted into a pool of unique acceptor plasmids after a AAV rep gene and before an ITR, a PolIII promoter, a unique barcode, a PolII promoter, GFP, and another ITR. This library of barcoded capsids is split into two fractions. One fraction is used for library delivery, while the second fraction of the library is preserved for viral gene identification as described below.

### Example 2

### Library Delivery

Four adults (8 weeks of age) C57/BL6 male mice are selected as hosts for the viral library screen. The viral library is diluted in 1×PBS, to a final titer of 10\hat{ }11 viral genomes per 50 uL. After anesthetization using isoflurane, the virus is delivered by instillation. The mice are observed after waking from anesthesia, and the following morning, to ensure that no adverse reaction to the viral delivery occurs.

### Example 3

### Cell Isolation and Sequencing

The first host mouse is sacrificed after a 4-week incubation period to allow expression of the library.

First, a dissociation solution is prepared: The following enzymes are dissolved in 5 mL DMEM/F12 (DFL3) (Caisson Labs): 13 mg lyophilized Collagenase I (Thermo Fisher), 50 mg lyophilized Dispase II (Sigma-Aldrich), 0.1% v/v elastase (Worthington), 1.25 mg DNase I (Sigma-Aldrich).

The host mouse as well as a noninjected mouse are sequentially anesthetized using isoflurane, sterilized with ethanol, and the abdominal cavity surgically opened to remove lungs. Ribs are removed to access lungs. Lungs are perfused with cold PBS, then 1 mL dissociation solution is injected through the trachea, and the trachea held closed with a hemostat for 60 seconds. The entire lung is resected into a petri dish, where lobes are removed from airway tissue and sliced into <2 mm pieces. Lung pieces are transferred to the rest of the dissociation fluid for 30 minutes of incubation at 37 degrees. At this point, and every 10 minutes thereafter, an aliquot of the cell suspension is taken for quantification. Cell suspension is mixed 1:1 with Trypan Blue Stain 0.4% (Thermo Fisher), and the number of cells and live cell percentage quantified using a Countess II (Thermo Fisher). When the number of live cells in suspension stops increasing, the cell suspension is advanced to FACS.

Cell sorting is done on a FACS Aria2 (BD), using flow rate 6. The cell suspension produced by the noninjected mouse is used to cell gates that exclusive auto-fluorescent cells. After gates are set up, the cell suspension from the injected host mouse is sorted until 50,000 GFP positive cells have been collected. Collected cells are immediately loaded into a Chromium chip (10× Genomics) per manufacturer's protocol. The 10× barcoded GEMs are collected and turned into Illumina sequencing libraries per manufacturer's protocols. During this process, 25% of the GEM cDNA is separated and used to PCR amplify the variant barcodes prior to sequencing. 25 cycles of PCR amplification using Q5 polymerase and buffers (NEB), with primers in the PCR handle included in the virus-identifying barcode, and in the 10× barcode region attached to each piece of RNA by the Chromium. This is followed by 5 cycles using the same primers with Illumina P5 and P7 sequences attached, to enable next-generation sequencing.

The 10×GEM cDNA and amplified barcode cDNA are loaded (95:5 ratio) to an Illumina Nextseq, using a 75-cycle high output kit per manufacturer's instructions. Upon completion of the sequencing run, another identical sequencing run is performed to add read depth.

### Example 4

### Data Analysis

Raw sequencing data is processed using bcl2fastq software (Illumina), aligned using STAR, followed by CellRanger (10× Genomics) to assign reads to individual cells. Given inefficiencies in the single-cell sequencing workflow, around 50,000 cells are identified by sequencing. Cell types are clustered using scVI, based on annotation in. A custom tool then maps virus-identifying barcode reads to individual cells, based on 10× barcodes detected in those reads. This results in groups of cells identifiable as having received a specific delivery vehicle. Differential gene expression is compared across these groups to identify transcription effects of the delivery vehicle. This analysis is repeated with comparisons restricted to cells of the same type.

### Example 5

### Viral Gene Identification

An aliquot of the plasmid library saved prior to injection into the animal is used to isolate the cap-barcode pair(s) of interest. The region of interest from the AAV cap gene through the left ITR and to the virus-identifying barcode is amplified using 25 cycles of PCR amplification using Q5 polymerase and buffers (NEB). The amplicon pool is visualized on an agarose gel and products between 3500 to 4500 base pairs in length are extracted to generate a purified amplicon pool; sample concentration was measured by Qubit 1× dsDNA High-Sensitivity Assay. 2 μg of the purified amplicon pool is processed using the SMRTbell Express Template Prep Kit 2.0 to generate sequencing libraries compatible for circular consensus sequencing on a PacBio SequelII machine, as per manufacturer's instructions. The region of interest is sequenced using circular consensus sequencing in which the PacBio polymerase circled the insert at least ten times to generate reads of greater than 99.999% Q50 accuracy, and consensus sequences are returned in fastq format. The sequences are then processed with PacBio SMRT Analysis software and aligned to a reference sequence using pbmm2 to generate a look-up table that matches cap variants with a distinct barcode that is identified in example 4.

Although the invention has been described with reference to the above examples, it will be understood that modifications and variations are encompassed within the spirit and scope of the invention. Accordingly, the invention is limited only by the following claims.

SEQUENCES

| Name | Sequence | SEQ ID NO: |
|---|---|---|
| capture sequence | GCTTTAAGGCCGGTCCTAGCAA | SEQ ID NO: 1 |
| capture sequence | GCTCACCTATTAGCGGCTAAGG | SEQ ID NO: 2 |
| Spike oligonucleotide | AAGCAGTGGTATCAACGCAGAGTACCAAGTT | SEQ ID NO: 3 |
| | GATAACGGACTAGCC | |
| Spike oligonucleotide | AAGCAGTGGTATCAACGCAGAGTACTTGCTAG | SEQ ID NO: 4 |
| | GACCGGCCTTAAAGC | |
| molecular enrichment sequence | CTTGGATCGTACCGTACGAA | SEQ ID NO: 5 |
| molecular enrichment sequence | CCCCNN | SEQ ID NO: 6 |
| (N = A/T/C/G) | | |
| molecular enrichment sequence | NNCCCC | SEQ ID NO: 7 |
| (N = A/T/C/G) | | |
| molecular enrichment sequence | CCCCTCCCCCAACCCCCC | SEQ ID NO: 8 |
| molecular enrichment sequence | CCCCACCCCCACCCCCAT | SEQ ID NO: 9 |
| molecular enrichment sequence | CCCCTTCCCCGTCCCCGC | SEQ ID NO: 10 |
| molecular enrichment sequence | CCCCTTCCCCATCCCCCC | SEQ ID NO: 11 |
| molecular enrichment sequence | CCCCTGCCCCCACCCCCC | SEQ ID NO: 12 |
| molecular enrichment sequence | CCCCGTCCCCCCCCCCCG | SEQ ID NO: 13 |
| molecular enrichment sequence | CCCCTTCCCCGACCCCGA | SEQ ID NO: 14 |
| molecular enrichment sequence | CCCCTCCCCCTCCCCCGT | SEQ ID NO: 15 |
| molecular enrichment sequence | CCCCTACCCCGACCCCCG | SEQ ID NO: 16 |
| molecular enrichment sequence | CCCCGGCCCCGACCCCTG | SEQ ID NO: 17 |
| molecular enrichment sequence | CCCCTTCCCCAACCCCAT | SEQ ID NO: 18 |
| molecular enrichment sequence | CCCCGTCCCCGGCCCCGA | SEQ ID NO: 19 |
| molecular enrichment sequence | CCCCGACCCCGACCCCAT | SEQ ID NO: 20 |
| molecular enrichment sequence | CCCCTCCCCCTTCCCCAC | SEQ ID NO: 21 |
| molecular enrichment sequence | CCCCGGCCCCTTCCCCCT | SEQ ID NO: 22 |
| molecular enrichment sequence | CCCCAGCCCCTCCCCCAT | SEQ ID NO: 23 |
| molecular enrichment sequence | CCCCTTCCCCTACCCCCT | SEQ ID NO: 24 |
| molecular enrichment sequence | CCCCATCCCCTGCCCCCC | SEQ ID NO: 25 |
| molecular enrichment sequence | CCCCTTCCCCCGCCCCGT | SEQ ID NO: 26 |
| molecular enrichment sequence | CCCCCTCCCCACCCCCGA | SEQ ID NO: 27 |
| molecular enrichment sequence | CCCCCGCCCCGCCCCCGT | SEQ ID NO: 28 |
| molecular enrichment sequence | CCCCGGCCCCATCCCCAC | SEQ ID NO: 29 |
| molecular enrichment sequence | CCCCCCCCCGACCCCCC | SEQ ID NO: 30 |
| molecular enrichment sequence | CCCCTCCCCCAACCCCCC | SEQ ID NO: 31 |
| molecular enrichment sequence | CCCCACCCCCACCCCCAT | SEQ ID NO: 32 |
| molecular enrichment sequence | CCCCTTCCCCGTCCCCGC | SEQ ID NO: 33 |
| molecular enrichment sequence | CCCCTTCCCCATCCCCCC | SEQ ID NO: 34 |
| molecular enrichment sequence | CCCCTGCCCCCACCCCCC | SEQ ID NO: 35 |
| molecular enrichment sequence | CCCCGTCCCCCCCCCCCG | SEQ ID NO: 36 |
| molecular enrichment sequence | CCCCTTCCCCGACCCCGA | SEQ ID NO: 37 |
| molecular enrichment sequence | CCCCTCCCCCTCCCCCGT | SEQ ID NO: 38 |
| molecular enrichment sequence | CCCCTACCCCGACCCCCG | SEQ ID NO: 39 |
| molecular enrichment sequence | CCCCGGCCCCGACCCCTG | SEQ ID NO: 40 |
| molecular enrichment sequence | CCCCTTCCCCAACCCCAT | SEQ ID NO: 41 |
| molecular enrichment sequence | CCCCGTCCCCGGCCCCGA | SEQ ID NO: 42 |
| molecular enrichment sequence | CCCCGACCCCGACCCCAT | SEQ ID NO: 43 |
| molecular enrichment sequence | CCCCTCCCCCTTCCCCAC | SEQ ID NO: 44 |
| molecular enrichment sequence | CCCCGGCCCCTTCCCCCT | SEQ ID NO: 45 |
| molecular enrichment sequence | CCCCAGCCCCTCCCCCAT | SEQ ID NO: 46 |
| molecular enrichment sequence | CCCCTTCCCCTACCCCCT | SEQ ID NO: 47 |
| molecular enrichment sequence | CCCCATCCCCTGCCCCCC | SEQ ID NO: 48 |
| molecular enrichment sequence | CCCCTTCCCCCGCCCCGT | SEQ ID NO: 49 |
| molecular enrichment sequence | CCCCCTCCCCACCCCCGA | SEQ ID NO: 50 |
| molecular enrichment sequence | CCCCCGCCCCGCCCCCGT | SEQ ID NO: 51 |
| molecular enrichment sequence | CCCCGGCCCCATCCCCAC | SEQ ID NO: 52 |
| molecular enrichment sequence | CCCCACCCCCGACCCCCC | SEQ ID NO: 53 |
| molecular enrichment sequence | CACCCCCCCCCCATCCCC | SEQ ID NO: 54 |
| molecular enrichment sequence | GGACCTTGCCTTGGATTGGA | SEQ ID NO: 55 |
| molecular enrichment sequence | GACCGAGGTGTTGGACGTTT | SEQ ID NO: 56 |
| molecular enrichment sequence | GGACCGCGGTAGCAGTACCG | SEQ ID NO: 57 |
| molecular enrichment sequence | GGCCATATGGTTTGCAAGTT | SEQ ID NO: 58 |
| molecular enrichment sequence | GGACCATGAGAGGGCACGAT | SEQ ID NO: 59 |
| molecular enrichment sequence | GGCCCTAGGCAGTGCTGCGG | SEQ ID NO: 60 |
| molecular enrichment sequence | GAGCCTTGGCTTAGGTACCG | SEQ ID NO: 61 |
| molecular enrichment sequence | ATGCTTGGACTGTATCGATA | SEQ ID NO: 62 |
| molecular enrichment sequence | GCTGACTGGCTGTTTGTAGT | SEQ ID NO: 63 |
| molecular enrichment sequence | GCTTGGACTGTACTTAAGGT | SEQ ID NO: 64 |
| molecular enrichment sequence | GGACTGTGTCTCTCATAGCA | SEQ ID NO: 65 |
| molecular enrichment sequence | GGACCGTGGCTGTAGTCGTA | SEQ ID NO: 66 |
| molecular enrichment sequence | GACCTCATGTCGCGTTGCTT | SEQ ID NO: 67 |
| molecular enrichment sequence | GACACAAGGCCTGCATATTT | SEQ ID NO: 68 |
| molecular enrichment sequence | GGACCGAGAACGTTTTCTGC | SEQ ID NO: 69 |
| molecular enrichment sequence | GGACCATCCTGTGCACGGGC | SEQ ID NO: 70 |
| molecular enrichment sequence | GGCCGCGCTTTGCGTGTCGA | SEQ ID NO: 71 |
| molecular enrichment sequence | CTTGGACTCTATGTAATAAT | SEQ ID NO: 72 |
| molecular enrichment sequence | GACCTGGTGTAGGGGTTGTC | SEQ ID NO: 73 |
| molecular enrichment sequence | GGACTTGGGCTTGATCTGCA | SEQ ID NO: 74 |
| molecular enrichment sequence | ACCTATGGCCCAACTAGCTA | SEQ ID NO: 75 |
| molecular enrichment sequence | GGGCTGTGCCTAGTGCGTTT | SEQ ID NO: 76 |
| molecular enrichment sequence | GACCCGGTAGGATTGTCTTT | SEQ ID NO: 77 |
| molecular enrichment sequence | GACTCGTCCTGAGGCATACA | SEQ ID NO: 78 |
| molecular enrichment sequence | GACCTTCTTGTGTATGAGGT | SEQ ID NO: 79 |
| molecular enrichment sequence | GGCCCCTTATGGTTCTAGTC | SEQ ID NO: 80 |
| molecular enrichment sequence | GGATTCGGCAAAAGGAATGG | SEQ ID NO: 81 |
| molecular enrichment sequence | GACCTTCTTGTGTATGAGGT | SEQ ID NO: 82 |
| molecular enrichment sequence | GGCCCCTTATGGTTCTAGTC | SEQ ID NO: 83 |
| molecular enrichment sequence | GGATTCGGCAAAAGGAATGG | SEQ ID NO: 84 |
| UGI sequence (N = A/T/C/G; | NNVNVNVN | SEQ ID NO: 85 |
| V = A/C/G) | | |

1. A method for identifying a vehicle effective in targeting a particular cell type comprising:
a) administering to an animal or an organoid a library comprising two or more distinct delivery vehicles, each delivery vehicle comprising:
i) distinct variant of a virus;
ii) a nucleic acid sequence encoding a distinct virus-identifying barcode region specific for each of the virus variants, wherein the barcode sequence is different than a nucleic acid sequence encoding a protein of the variant of the virus; and
iii) a nucleic acid sequence encoding at least one reporter, which when expressed in a cell, is indicative of a cell state or a likelihood of a cell state of a cell;
b) obtaining a sample from the animal or organoid to generate a cell population;
c) enriching the cell population for those cells containing a reporter;
d) using single cell sequencing to identify a delivery vehicle that results in a change in a cell state or a likelihood of a cell state of a cell of the animal or the organoid and thereby the vector; and
e) using single cell sequencing to identify the type of cells having the change in cell state and to determine the relative rate of transduction for one of the distinct vectors in the different cell types, thereby identifying the vehicle.

2-4. (canceled)

5. The method of claim 1, wherein the change in cell state or likelihood of a change in cell state indicates the successful delivery and expression of the nucleic acid sequences to a cell of the cell population after enriching.

6. The method of claim 5, wherein the cell state or likelihood of cell state is determined by the presence of increased or decreased levels of proteins or nucleic acid sequences.

7. (canceled)

8. The method of claim 1, wherein identifying comprises identifying the delivery vehicle based on the presence of a reporter and vector-identifying barcode within a cell of the cell population after enriching.

9. The method of claim 8, wherein the identification step further comprises identifying the cell type of a cell determined to have been effected by the delivery vehicle.

10-28. (canceled)

29. A library comprising two or more distinct delivery vehicles, each delivery vehicle comprising:
a) a distinct variant of a virus;
b) a nucleic acid sequence encoding a distinct virus-identifying barcode region specific for each of the virus variants, wherein the barcode sequence is different than a nucleic acid sequence encoding a protein of the variant of the virus; and
c) a nucleic acid sequence encoding at least one reporter, which when expressed in a cell, is indicative of a cell state or a likelihood of a cell state of a cell.

30. (canceled)

31. The library of claim 29, wherein each of the vectors are selected from the group consisting of adeno-associated viruses and lentivirus.

32. The library of claim 29, wherein the distinct variants of a virus are substituted for distinct variants of lipid nanoparticles.

33. (canceled)

34. The library of claim 29, wherein the distinct variant of a virus contains a uniquely modified cap gene region linked to the distinct virus-identifying barcode region.

35. The library of claim 34, wherein the cap gene and distinct virus-identifying barcode regions are isolated using beads affixed with complementary DNA to the distinct virus-identifying barcode region.

36. The library of claim 35, wherein the regions that were isolated are identified by
insertion of the region into a new plasmid;
amplification of the new plasmid; and
Sanger sequencing of the plasmid.

37. The library of claim 29, wherein a Polymerase III promotor region is operably linked to the distinct virus-identifying barcode region.

38. The library of claim 37, wherein a capture sequence having a sequence comprising any one of SEQ ID NOs:1-4 is operably linked to the distinct virus-identifying barcode region under the control of the Polymerase III promoter.

39. (canceled)

40. The library of claim 37, wherein one or more molecular enrichment sequences having a sequence comprising any one of SEQ ID NOs:5-84 are operably linked to the distinct virus-identifying barcode region under the control of the Polymerase III promoter.

41. (canceled)

42. The library of claim 37, wherein a unique genome identification (UGI) sequence is operably linked to the distinct virus-identifying barcode region under the control of the Polymerase III promoter.

43. The library of claim 43, wherein the UGI has a sequence comprising SEQ ID NO:85.

44. The library of claim 29, wherein the library comprises about 5-10,000,000 or more distinct delivery vehicles.

45-49. (canceled)

50. A method for identifying a vehicle effective in targeting a particular cell type comprising:
a) administering to an animal or an organoid a library comprising two or more distinct delivery vehicles, each delivery vehicle comprising:
i) a distinct variant of an adeno-associated virus;
ii) a nucleic acid sequence encoding a distinct virus-identifying barcode region specific for each of the virus variants, wherein the barcode sequence is different than a nucleic acid sequence encoding a protein of the variant of the virus; and
iii) a nucleic acid sequence encoding GFP, which when expressed in a cell, is indicative of successful delivery of the nucleic acid sequences to a cell;
b) obtaining a sample from the animal or organoid to generate a cell population;
c) enriching the cell population for those cells containing GFP;
d) using single cell sequencing to identify a delivery vehicle that results in expression of the nucleic acid sequences with a cell; and
e) using single cell sequencing to identify the type of cells having the change in cell state and to determine the relative rate of transduction for one of the distinct vectors in the different cell types, thereby identifying the vehicle.

51. The method of claim 50, further comprising identifying a type of transduced cell, and/or a localization of a transduced cell in a tissue.

52. The method of claim 50, wherein identifying comprises using spatial transcriptomics.


SEQUENCE LISTING (85 sequences)

SEQ ID NO: 1 - DNA, 22 bp, Artificial Sequence
  Description of Artificial Sequence Synthetic oligonucleotide
gctttaaggccggtcctagcaa

SEQ ID NO: 2 - DNA, 22 bp, Artificial Sequence
  Description of Artificial Sequence Synthetic oligonucleotide
gctcacctattagcggctaagg

SEQ ID NO: 3 - DNA, 46 bp, Artificial Sequence
  Description of Artificial Sequence Synthetic oligonucleotide
aagcagtggtatcaacgcagagtaccaagttgataacggactagcc

SEQ ID NO: 4 - DNA, 47 bp, Artificial Sequence
  Description of Artificial Sequence Synthetic oligonucleotide
aagcagtggtatcaacgcagagtacttgctaggaccggccttaaagc

SEQ ID NO: 5 - DNA, 20 bp, Artificial Sequence
  Description of Artificial Sequence Synthetic oligonucleotide
cttggatcgtaccgtacgaa

SEQ ID NO: 6 - DNA, 6 bp, Artificial Sequence
  Description of Artificial Sequence Synthetic oligonucleotide
  modified_base (5)..(6): a, t, c or g
ccccnn

SEQ ID NO: 7 - DNA, 6 bp, Artificial Sequence
  Description of Artificial Sequence Synthetic oligonucleotide
  modified_base (1)..(2): a, t, c or g
nncccc

SEQ ID NO: 8 - DNA, 18 bp, Artificial Sequence
  Description of Artificial Sequence Synthetic oligonucleotide
cccctcccccaacccccc

SEQ ID NO: 9 - DNA, 18 bp, Artificial Sequence
  Description of Artificial Sequence Synthetic oligonucleotide
ccccacccccacccccat

SEQ ID NO: 10 - DNA, 18 bp, Artificial Sequence
  Description of Artificial Sequence Synthetic oligonucleotide
ccccttccccgtccccgc

SEQ ID NO: 11 - DNA, 18 bp, Artificial Sequence
  Description of Artificial Sequence Synthetic oligonucleotide
ccccttccccatcccccc

SEQ ID NO: 12 - DNA, 18 bp, Artificial Sequence
  Description of Artificial Sequence Synthetic oligonucleotide
cccctgcccccacccccc

SEQ ID NO: 13 - DNA, 18 bp, Artificial Sequence
  Description of Artificial Sequence Synthetic oligonucleotide
ccccgtcccccccccccg

SEQ ID NO: 14 - DNA, 18 bp, Artificial Sequence
  Description of Artificial Sequence Synthetic oligonucleotide
ccccttccccgaccccga

SEQ ID NO: 15 - DNA, 18 bp, Artificial Sequence
  Description of Artificial Sequence Synthetic oligonucleotide
cccctccccctcccccgt

SEQ ID NO: 16 - DNA, 18 bp, Artificial Sequence
  Description of Artificial Sequence Synthetic oligonucleotide
cccctaccccgacccccg

SEQ ID NO: 17 - DNA, 18 bp, Artificial Sequence
  Description of Artificial Sequence Synthetic oligonucleotide
ccccggccccgacccctg

SEQ ID NO: 18 - DNA, 18 bp, Artificial Sequence
  Description of Artificial Sequence Synthetic oligonucleotide
ccccttccccaaccccat

SEQ ID NO: 19 - DNA, 18 bp, Artificial Sequence
  Description of Artificial Sequence Synthetic oligonucleotide
ccccgtccccggccccga

SEQ ID NO: 20 - DNA, 18 bp, Artificial Sequence
  Description of Artificial Sequence Synthetic oligonucleotide
ccccgaccccgaccccat

SEQ ID NO: 21 - DNA, 18 bp, Artificial Sequence
  Description of Artificial Sequence Synthetic oligonucleotide
cccctcccccttccccac

SEQ ID NO: 22 - DNA, 18 bp, Artificial Sequence
  Description of Artificial Sequence Synthetic oligonucleotide
ccccggccccttccccct

SEQ ID NO: 23 - DNA, 18 bp, Artificial Sequence
  Description of Artificial Sequence Synthetic oligonucleotide
ccccagcccctcccccat

SEQ ID NO: 24 - DNA, 18 bp, Artificial Sequence
  Description of Artificial Sequence Synthetic oligonucleotide
ccccttcccctaccccct

SEQ ID NO: 25 - DNA, 18 bp, Artificial Sequence
  Description of Artificial Sequence Synthetic oligonucleotide
ccccatcccctgcccccc

SEQ ID NO: 26 - DNA, 18 bp, Artificial Sequence
  Description of Artificial Sequence Synthetic oligonucleotide
ccccttcccccgccccgt

SEQ ID NO: 27 - DNA, 18 bp, Artificial Sequence
  Description of Artificial Sequence Synthetic oligonucleotide
ccccctccccacccccga

SEQ ID NO: 28 - DNA, 18 bp, Artificial Sequence
  Description of Artificial Sequence Synthetic oligonucleotide
cccccgccccgcccccgt

SEQ ID NO: 29 - DNA, 18 bp, Artificial Sequence
  Description of Artificial Sequence Synthetic oligonucleotide
ccccggccccatccccac

SEQ ID NO: 30 - DNA, 17 bp, Artificial Sequence
  Description of Artificial Sequence Synthetic oligonucleotide
cccccccccgacccccc

SEQ ID NO: 31 - DNA, 18 bp, Artificial Sequence
  Description of Artificial Sequence Synthetic oligonucleotide
cccctcccccaacccccc

SEQ ID NO: 32 - DNA, 18 bp, Artificial Sequence
  Description of Artificial Sequence Synthetic oligonucleotide
ccccacccccacccccat

SEQ ID NO: 33 - DNA, 18 bp, Artificial Sequence
  Description of Artificial Sequence Synthetic oligonucleotide
ccccttccccgtccccgc

SEQ ID NO: 34 - DNA, 18 bp, Artificial Sequence
  Description of Artificial Sequence Synthetic oligonucleotide
ccccttccccatcccccc

SEQ ID NO: 35 - DNA, 18 bp, Artificial Sequence
  Description of Artificial Sequence Synthetic oligonucleotide
cccctgcccccacccccc

SEQ ID NO: 36 - DNA, 18 bp, Artificial Sequence
  Description of Artificial Sequence Synthetic oligonucleotide
ccccgtcccccccccccg

SEQ ID NO: 37 - DNA, 18 bp, Artificial Sequence
  Description of Artificial Sequence Synthetic oligonucleotide
ccccttccccgaccccga

SEQ ID NO: 38 - DNA, 18 bp, Artificial Sequence
  Description of Artificial Sequence Synthetic oligonucleotide
cccctccccctcccccgt

SEQ ID NO: 39 - DNA, 18 bp, Artificial Sequence
  Description of Artificial Sequence Synthetic oligonucleotide
cccctaccccgacccccg

SEQ ID NO: 40 - DNA, 18 bp, Artificial Sequence
  Description of Artificial Sequence Synthetic oligonucleotide
ccccggccccgacccctg

SEQ ID NO: 41 - DNA, 18 bp, Artificial Sequence
  Description of Artificial Sequence Synthetic oligonucleotide
ccccttccccaaccccat

SEQ ID NO: 42 - DNA, 18 bp, Artificial Sequence
  Description of Artificial Sequence Synthetic oligonucleotide
ccccgtccccggccccga

SEQ ID NO: 43 - DNA, 18 bp, Artificial Sequence
  Description of Artificial Sequence Synthetic oligonucleotide
ccccgaccccgaccccat

SEQ ID NO: 44 - DNA, 18 bp, Artificial Sequence
  Description of Artificial Sequence Synthetic oligonucleotide
cccctcccccttccccac

SEQ ID NO: 45 - DNA, 18 bp, Artificial Sequence
  Description of Artificial Sequence Synthetic oligonucleotide
ccccggccccttccccct

SEQ ID NO: 46 - DNA, 18 bp, Artificial Sequence
  Description of Artificial Sequence Synthetic oligonucleotide
ccccagcccctcccccat

SEQ ID NO: 47 - DNA, 18 bp, Artificial Sequence
  Description of Artificial Sequence Synthetic oligonucleotide
ccccttcccctaccccct

SEQ ID NO: 48 - DNA, 18 bp, Artificial Sequence
  Description of Artificial Sequence Synthetic oligonucleotide
ccccatcccctgcccccc

SEQ ID NO: 49 - DNA, 18 bp, Artificial Sequence
  Description of Artificial Sequence Synthetic oligonucleotide
ccccttcccccgccccgt

SEQ ID NO: 50 - DNA, 18 bp, Artificial Sequence
  Description of Artificial Sequence Synthetic oligonucleotide
ccccctccccacccccga

SEQ ID NO: 51 - DNA, 18 bp, Artificial Sequence
  Description of Artificial Sequence Synthetic oligonucleotide
cccccgccccgcccccgt

SEQ ID NO: 52 - DNA, 18 bp, Artificial Sequence
  Description of Artificial Sequence Synthetic oligonucleotide
ccccggccccatccccac

SEQ ID NO: 53 - DNA, 18 bp, Artificial Sequence
  Description of Artificial Sequence Synthetic oligonucleotide
ccccacccccgacccccc

SEQ ID NO: 54 - DNA, 18 bp, Artificial Sequence
  Description of Artificial Sequence Synthetic oligonucleotide
caccccccccccatcccc

SEQ ID NO: 55 - DNA, 20 bp, Artificial Sequence
  Description of Artificial Sequence Synthetic oligonucleotide
ggaccttgccttggattgga

SEQ ID NO: 56 - DNA, 20 bp, Artificial Sequence
  Description of Artificial Sequence Synthetic oligonucleotide
gaccgaggtgttggacgttt

SEQ ID NO: 57 - DNA, 20 bp, Artificial Sequence
  Description of Artificial Sequence Synthetic oligonucleotide
ggaccgcggtagcagtaccg

SEQ ID NO: 58 - DNA, 20 bp, Artificial Sequence
  Description of Artificial Sequence Synthetic oligonucleotide
ggccatatggtttgcaagtt

SEQ ID NO: 59 - DNA, 20 bp, Artificial Sequence
  Description of Artificial Sequence Synthetic oligonucleotide
ggaccatgagagggcacgat

SEQ ID NO: 60 - DNA, 20 bp, Artificial Sequence
  Description of Artificial Sequence Synthetic oligonucleotide
ggccctaggcagtgctgcgg

SEQ ID NO: 61 - DNA, 20 bp, Artificial Sequence
  Description of Artificial Sequence Synthetic oligonucleotide
gagccttggcttaggtaccg

SEQ ID NO: 62 - DNA, 20 bp, Artificial Sequence
  Description of Artificial Sequence Synthetic oligonucleotide
atgcttggactgtatcgata

SEQ ID NO: 63 - DNA, 20 bp, Artificial Sequence
  Description of Artificial Sequence Synthetic oligonucleotide
gctgactggctgtttgtagt

SEQ ID NO: 64 - DNA, 20 bp, Artificial Sequence
  Description of Artificial Sequence Synthetic oligonucleotide
gcttggactgtacttaaggt

SEQ ID NO: 65 - DNA, 20 bp, Artificial Sequence
  Description of Artificial Sequence Synthetic oligonucleotide
ggactgtgtctctcatagca

SEQ ID NO: 66 - DNA, 20 bp, Artificial Sequence
  Description of Artificial Sequence Synthetic oligonucleotide
ggaccgtggctgtagtcgta

SEQ ID NO: 67 - DNA, 20 bp, Artificial Sequence
  Description of Artificial Sequence Synthetic oligonucleotide
gacctcatgtcgcgttgctt

SEQ ID NO: 68 - DNA, 20 bp, Artificial Sequence
  Description of Artificial Sequence Synthetic oligonucleotide
gacacaaggcctgcatattt

SEQ ID NO: 69 - DNA, 20 bp, Artificial Sequence
  Description of Artificial Sequence Synthetic oligonucleotide
ggaccgagaacgttttctgc

SEQ ID NO: 70 - DNA, 20 bp, Artificial Sequence
  Description of Artificial Sequence Synthetic oligonucleotide
ggaccatcctgtgcacgggc

SEQ ID NO: 71 - DNA, 20 bp, Artificial Sequence
  Description of Artificial Sequence Synthetic oligonucleotide
ggccgcgctttgcgtgtcga

SEQ ID NO: 72 - DNA, 20 bp, Artificial Sequence
  Description of Artificial Sequence Synthetic oligonucleotide
cttggactctatgtaataat

SEQ ID NO: 73 - DNA, 20 bp, Artificial Sequence
  Description of Artificial Sequence Synthetic oligonucleotide
gacctggtgtaggggttgtc

SEQ ID NO: 74 - DNA, 20 bp, Artificial Sequence
  Description of Artificial Sequence Synthetic oligonucleotide
ggacttgggcttgatctgca

SEQ ID NO: 75 - DNA, 20 bp, Artificial Sequence
  Description of Artificial Sequence Synthetic oligonucleotide
acctatggcccaactagcta

SEQ ID NO: 76 - DNA, 20 bp, Artificial Sequence
  Description of Artificial Sequence Synthetic oligonucleotide
gggctgtgcctagtgcgttt

SEQ ID NO: 77 - DNA, 20 bp, Artificial Sequence
  Description of Artificial Sequence Synthetic oligonucleotide
gacccggtaggattgtcttt

SEQ ID NO: 78 - DNA, 20 bp, Artificial Sequence
  Description of Artificial Sequence Synthetic oligonucleotide
gactcgtcctgaggcataca

SEQ ID NO: 79 - DNA, 20 bp, Artificial Sequence
  Description of Artificial Sequence Synthetic oligonucleotide
gaccttcttgtgtatgaggt

SEQ ID NO: 80 - DNA, 20 bp, Artificial Sequence
  Description of Artificial Sequence Synthetic oligonucleotide
ggccccttatggttctagtc

SEQ ID NO: 81 - DNA, 20 bp, Artificial Sequence
  Description of Artificial Sequence Synthetic oligonucleotide
ggattcggcaaaaggaatgg

SEQ ID NO: 82 - DNA, 20 bp, Artificial Sequence
  Description of Artificial Sequence Synthetic oligonucleotide
gaccttcttgtgtatgaggt

SEQ ID NO: 83 - DNA, 20 bp, Artificial Sequence
  Description of Artificial Sequence Synthetic oligonucleotide
ggccccttatggttctagtc

SEQ ID NO: 84 - DNA, 20 bp, Artificial Sequence
  Description of Artificial Sequence Synthetic oligonucleotide
ggattcggcaaaaggaatgg

SEQ ID NO: 85 - DNA, 8 bp, Artificial Sequence
  Description of Artificial Sequence Synthetic oligonucleotide
  modified_base (1)..(2): a, t, c or g
  modified_base (4)..(4): a, t, c or g
  modified_base (6)..(6): a, t, c or g
  modified_base (8)..(8): a, t, c or g
nnvnvnvn